(19) United States
(12) Patent Application Publication
(10) Pub. No.: US 2005/0203150 A1
(43) Pub. Date: Sep. 15, 2005

(54) Compounds, compositions and methods for inhibiting or treating HIV-1

(76) Inventors: Omar K. Haffar, Seattle, WA (US); Jeffrey W. Godden, Seattle, WA (US); Jurgen Bajorath, Lynnwood, WA (US)

Correspondence Address: Supervisor, Patent Prosecution Services;PIPER RUDNICK LLP, 1200 Nineteenth Street, N.W., Washington, DC, 20036-2412, US

(21) Appl. No.: 10/795,505

(22) Filed: Mar. 9, 2004

(51) Int. Cl. A61K 31/4245; C07D 271/12

(52) U.S. Cl. 514/364; 548/132

(57) ABSTRACT

There is disclosed a structural genus of compounds, defined according to coordinates in three-dimensional space, that bind amino moieties on neighboring residues in a tyrosine residue 29 pocket of the matrix protein component of the HIV-1 preintegration complex (PIC), thereby preventing the PIC from binding to karyopherin α and preventing nuclear importation and integration of the HIV-1 viral genome into the host cell DNA, thereby preventing viral infection. The compounds may be utilized alone or in combinations with known inhibitors for preventing or inhibiting HIV-1 infection.

This invention was made with Government support under Grant Number 2R44AI47782-02 awarded by the National Institutes of Health as a Small Business Innovative Research-Advanced Technology Grant from the National Institute of Allergy and Infectious Diseases. The Government may have certain rights in the invention.

### BACKGROUND OF THE INVENTION

1. Field of the Invention

The present invention is directed to compounds, compositions, methods of making and methods of using products in the fields of pharmacology and immunology.

In one aspect the invention is directed to a structural genus of compounds. The structural genus is defined according to coordinates in a specific three-dimensional space, allowing binding by a compound to amino acid moieties forming that space according to the predicted structure of the HIV-1 matrix protein when in the in vivo conformation.

More particularly, the invention is directed to a genus of compounds that bind amino acid moieties that occur within a structural groove in the N-terminal Nuclear Localization Sequence (NLS). The structural groove is generated by the inward orientation of the tyrosine residue at position 29 of the HIV-1 matrix protein. The HIV-1 matrix protein is a component of the HIV preintegration complex (PIC).

The compounds according to the invention occupy the structural groove, thereby preventing or inhibiting the PIC from binding to karyopherin a and preventing or inhibiting nuclear importation and therefore subsequent integration of the HIV viral genome into the host cell DNA, thereby preventing or inhibiting viral infection and or replication.

2. Related Art

U.S. Pat. No. 5,808,068 to Pan et al. discloses compounds having anti-HIV therapeutic activity that inhibit nuclear localization of the HIV preintegration complex.

U.S. Pat. No. 5,849,793 to Pan et al. discloses compounds for binding inside the HIV Matrix Protein tyrosine position 29 pocket to inhibit PIC binding to karyopherin a thereby preventing nuclear importation and integration of the HIV viral genome into host cell DNA, thereby preventing viral infection.

U.S. Pat. No. 5,877,282 to Nadler et al. discloses polypeptide inhibitors of cytoplasmic protein nuclear translocation. The inhibitors comprise a signal sequence and a plurality of nuclear localization sequences. The polypeptides have applications in immuno-suppression, and as antiviral and anti-tumor agents.

U.S. Pat. No. 6,297,253 to Bukrinsky et al. discloses compounds used to target specific nuclear localization signals, thereby blocking importation of specific proteins or a molecular complex into the nucleus of a cell. Also disclosed are methods of using such compounds for treatment or prevention of infectious diseases, such as parasitic and viral diseases, including, for example, malaria and acquired immunodeficiency syndrome. The use of the compounds to detect certain specific protein structures which are present in nuclear localization sequences is also taught.

All documents cited herein are incorporated by reference for all purposes.

### BACKGROUND OF THE INVENTION

Human immunodeficiency virus type-1 (HIV-1) and other lentiviruses infect non-dividing terminally differentiated cells such as primary macrophages (Gendelman et al., J. Virol. 58:67-74, 1986; Gartner et al., Science 233:215-219, 1986), primary blood dendritic cells (Langhoffet al., Proc. Natl. Acad. Sci. USA 88:998-8002, 1991), and epidermal Langerhan's cells (Ramazzotti et al., Immunology 85:94-98, 1995). This is facilitated by the active importation of the HIV-1 preintegration complex (PIC), which incorporates the viral genome, across the intact nuclear envelope of the non-dividing cell (Bukrinsky et al., Proc. Natl. Acad. Sci. USA 89:6580-6584, 1992; Bukrinsky et al., Nature 365:666-669, 1993; and von Schwedler et al., Proc. Natl. Acad. Sci. USA 91:6992-6996, 1994). In addition, HIV-1 can establish productive infection in activated primary T cells at all steps of the cell cycle, prior to and including the M phase, when dissolution of the nuclear envelope occurs. Thus, active nuclear importation obviates the requirement for cell division, thus allowing HIV-1 to infect non-proliferating as well as proliferating cells (Lewis et al., EMBO J. 11:3053-3058, 1992), the usual targets of retroviruses (Roe et al., EMBO J. 12:2099-2108, 1993; and Lewis and Emerman, J. Virol. 68:510-516, 1994).

In addition to the viral genomic RNA, the PIC is composed of the gag-derived matrix protein (MA) and the nucleocapsid protein (NC), the reverse transcriptase (RT), integrase (IN), and Virus protein R (Vpr). Reverse transcription and production of the nascent cDNA is completed in the context of the PIC in the cytoplasm of a target cell, prior to nuclear entry. It has been shown (Gallay et al., J. Virol. 70:1027-1032, 1996; and Popov et al., Proc. Natl. Acad. Sci. USA 93:11859-11864, 1996) that the PIC of HIV-1 associates with karyopherins, the cellular proteins involved in active nuclear importation (reviewed in Adam, Trends Cell Biol. 5:189-191, 1995). Karyopherin α binds to target proteins via their nuclear localization sequence (NLS), while karyopherin β mediates docking of the karyopherin a-target protein complex to nuclear pore structures (Radu et al., Proc. Natl. Acad. Sci. USA 92:1769-1773, 1995; Moroianu et al., Proc. Natl. Acad. Sci USA 92:2008-2011, 1995; Gorlich et al., Nature (London) 377:246-248, 1995; Adam and Gerace, Cell 66:837-847, 1991; Gorlich and Mattaj, Science 271:1513-1518, 1996; and Hurt, Cell 84:509-515, 1996).

HIV-1 matrix protein (MA) contains two nuclear localization sequences. The first, MA NLS-1 is defined at (K²⁶ KKYK) and the second MA NLS-2 is found at (K¹¹⁰ SKKK). MA represents a major karyophilic structure within the PIC (Bukrinsky et al., Nature 365:666-669, 1993; von Schwedler et al., Proc. Natl. Acad. Sci. USA 91:6992-6996, 1994; Gallay et al., J. Virol. 70:1027-1032, 1996; and Bukrinsky et al., Proc. Natl. Acad. Sci. USA 90:6125-6129, 1993). Inactivation of both MA NLSs has been shown to preclude nuclear translocation of MA rendering the HIV-1 virus defective in nuclear import and replication in non-dividing macrophage cultures, even when functional Vpr and integrase were present. (Haffar et al., J. Mol. Biol. 299: 359-368, 2000).

Mutations in the K²⁶ KKYK (NLS-1 of MA), alone or in combination with the deletion of Vpr, reduced nuclear importation of the HIV-1 PIC and inhibited infection of primary macrophage cultures (von Schwedler et al., Proc. Natl. Acad. Sci. USA 91:6992-6996, 1994; Heizinger et al., Proc. Natl. Acad. Sci. USA 91:7311-7315, 1992), as well as growth-arrested T cells (Bukrinsky et al., Nature 365:666-669, 1993) and CD4⁺-HeLa cell cultures (Emerman et al., Nature (London) 369:107-108, 1994). Single amino acid substitutions within the K²⁶ KKYK of NLS-1 also reduced binding of the HIV-1 PIC to yeast karyopherin a in vitro (Popov et al., Proc. Natl. Acad. Sci. USA 93:11859-11864, 1996), thus providing a link between binding of PIC to karyopherin α, nuclear import, and viral replication in non-dividing cells.

Synthetic peptides encompassing either of the two MA nuclear localization sequences bound both identified human karyopherin α present in B cell and T cell lysates (Nadler et al., J. Biol. Chem. 272, 4310-4315, 1997). Linear peptides derived from the HIV-1 MA have been shown to inhibit HIV-1 replication in cultured cells. (Lanford et al., Cell 15: 575-582, 1986; Lanford et al., Mol. Cell Biol. 8:2722-2729, 1988; Dworetzky et al., J. Cell Biol. 106: 575-584, 1988; Adam et al., Cell 66:837-847, 1991). The inhibition of nuclear import has also been shown using cyclic petidomimetics derived from the HIV-1 MA NLS sequence, which function resembling receptor antagonists. (Hariton-Gazal et al., Bio. Biophysic. Acta 1584: 234-242, 2002). However, synthetic peptides are subject to proteolytic degradation, and thus more effective means are sought to inhibit or prevent HIV-1 infection.

### SUMMARY OF THE INVENTION

An object of the invention is providing a compound having the formula: 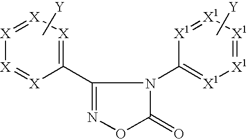
wherein X and X¹ are nitrogen or carbon insofar as at least three of X and at least three of X¹ are carbon; and when either X or X¹ are carbon, Y is covalently bonded to X or X^{1,} Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO₂, or —O—CH₃. The invention, as it relates to compounds is optionally described with respect to the general structure as a compound having the formula above but with the proviso that when A is a halogen, n is 0-2; or with the proviso that when at least one Y is halogen, the remaining of Y are not only either hydrogen or halogen; or with the proviso that when at least one Y contains a halogen, the remaining of Y are not only hydrogen; or with the proviso that all of Y are not only either hydrogen or alkyl; or with the proviso that when at least one Y is nitroxyl, the remaining of Y are not only hydrogen; or with the proviso that when at least one Y is an —O—R—(A)ₙ group, the remaining of Y are not only hydrogen. Also, all the specific compounds listed in Table 1 below can be excluded from the general formula above.

In another object, the invention is directed to a compound wherein X and X¹ are nitrogen or carbon insofar as at least three of X and at least three of X¹ are carbon; and when X or X¹ are carbon, Y is covalently bonded to X or X¹ and is either 1) hydrogen or 2) chlorine, fluorine, —NO₂, —CF₃, —CH₃, or —O—CH₃.

In yet another object, the invention is directed to uses of a compound, known as ITI-367, and having the formula:

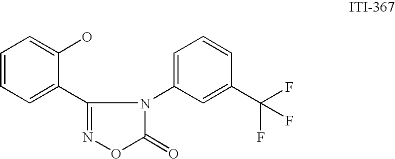

Another object of the invention is a pharmaceutical composition comprising such a compound and a pharmaceutical carrier.

Yet still another object of the invention is a method of making such a compound comprising synthesizing at least one compound having a formula according to one of the chemical structures above.

Another object of the invention is a method for treating HIV infection comprising administering an effective amount of at least one compound having a formula according to one of the chemical structures above.

Still another object of the invention is a combination therapeutic treatment regimen for the treatment of HIV infection, comprising a reverse transcriptase inhibitor and at least one compound having the formula according to one of the above chemical structures. In other objects the combination is one wherein the reverse transcriptase inhibitor is selected from the group consisting of 3TC, AZT, ddI, d4T, ddC, and combinations thereof. In other objects, the combination further comprises an HIV protease inhibitor. In still other objects the combination is one wherein the HIV protease inhibitor is selected from the group consisting of ritonavir, nelfinavir, saquinavir, indinavir, and combinations thereof.

Still other objects of the invention are combinations and method for treating HIV infection, comprising administering an effective amount of at least one compound having the formula according to one of the above chemical structures and another inhibitor. Inhibitors in these combinations and methods include Nucleoside analog Reverse Transcriptase inhibitors (NRTi), Non-Nucleoside analog Reverse Transcriptase inhibitors (NNRTi), Protease inhibitors (Pi), and Cell Entry inhibitors (Ci). Nucleoside analog Reverse Transcriptase inhibitor (NRTi) can include AZT, ZDV ddl, ddc, ddc, 3TC, and abacavir. Non-Nucleoside analog Reverse Transcriptase inhibitors (NNRTi) can include nevirapine, delavirdine, efavirenz, capravirine, and calanolide-A. Protease inhibitors (Pi) can include SQV, RTV, IDV, NFV, APV, LPV, ATZ, FPV, and TPV. Cell Entry inhibitors (Ci) can include Fuzeon, T-1249, PRO-542, and SCH-C.

In other objects the method is one wherein the reverse transcriptase inhibitor is selected from the group consisting of 3TC, AZT, ddI, d4T, ddC, and combinations thereof. In other objects, the method further comprises an HIV protease inhibitor. In still other objects the method is one wherein the HIV protease inhibitor is selected from the group consisting of ritonavir, nelfinavir, saquinavir, indinavir, and combinations thereof.

Yet another object of the invention is a method of making a pharmaceutical composition comprising combining a pharmaceutical carrier with at least one compound having a formula according to one of the above chemical structures, optionally in combination with at least one inhibitor.

### BRIEF DESCRIPTION OF THE INVENTION

The invention relates to compounds, compositions, methods of making these compounds and compositions and methods of using these compounds or compositions in treating or preventing HIV-1 infection. The compounds make up a structural genus predicted according to an algorithm defining coordinates in a specific three-dimensional space.

The structure of these compounds allows binding by these compounds to amino acid moieties forming that space according to the predicted structure of the HIV-1 matrix protein in the in vivo conformation, and more particularly, moieties that occur within a structural groove in the N-terminal Nuclear Localization Sequence (NLS). The structural groove is generated by the inward orientation of the tyrosine residue at position 29 of the HIV-1 matrix protein.

The HIV-1 matrix protein is a component of the HIV preintegration complex (PIC). The compounds according to the invention occupy the structural groove, thereby preventing the PIC from binding to karyopherin a and preventing nuclear importation and integration of the HIV viral genome into the host cell DNA, thereby preventing viral infection.

The compounds of the invention may be combined with inhibitors in combinations for therapy and in pharmaceutical compositions. Inhibitors in these combinations and methods include Nucleoside analog Reverse Transcriptase inhibitors (NRTi), Non-Nucleoside analog Reverse Transcriptase inhibitors (NNRTi), Protease inhibitors (Pi), and Cell Entry inhibitors (Ci).

### DESCRIPTION OF THE FIGURES

The foregoing summary, as well as the following detailed description, will be better understood when read in conjunction with the appended drawings. For the purpose of illustrating the invention, there is shown in the drawings embodiments which are presently preferred, it being understood that this invention is not limited to the precise arrangements and instrumentalities shown.

FIG. 1 is a graphical representation of the results of a cell free assay measuring the extent of binding of test compounds by evaluating the levels of HIV DNA found in the Kα fraction done in the presence or absence of a test compound.

FIG. 2 is a graphical representation of the results showing inhibition of HIV-1 infection by test compound ITI-367 in monocyte-derived macrophages over a two week period.

FIG. 3 is a graphical representation of the results showing inhibition of HIV-1 infection of T lymphocytes by test compound ITI-367 over a seventeen day period.

FIG. 4 is a graphical representation of the results of inhibition by ITI-367 upon replication of HIV-1 in Primary Lymphocytes.

FIG. 5 is a graphical representation showing the resulting effect of combining ITI-367 and the NRTi, AZT.

FIG. 6 is a graphical representation showing the resulting effect of combining ITI-367 and the NRTi, d4T.

FIG. 7 is a graphical representation showing the combination effect of ITI-367 and the Pi, Nelfinavir.

FIG. 8 is a graphical representation showing the combination effect of ITI-367 and the Pi, Saquinavir.

FIG. 9 is a graphical representation showing the combination effect of ITI-367 and the NNRTi, Efavirenz.

### DETAILED DESCRIPTION OF THE INVENTION

While this invention is satisfied by embodiments in many different forms, there will herein be described in detail preferred embodiments of the invention, with the understanding that the present disclosure is to be considered as exemplary of the principles of the invention and is not intended to limit the invention to the embodiments illustrated and described. Numerous variations may be made by persons skilled in the art without departure from the spirit of the invention.

Nuclear Entry is a requisite step in the life cycle of HIV-1 for generating a productive infection in target cells. This process is accomplished by the specific association of the viral pre-integration complex (PIC) carrying the nascent HIV-1 cDNA molecule, generated by reverse transcription of the packaged genomic RNA, with the intracellular protein karyopherin α. This specific association is mediated by the presence of nuclear localization sequences (NLSs) on the HIV-1 matrix protein present in the pre-integration complex. It has been previously shown that mutations within these NLSs or the use of small molecules that bind to amino acid residues that comprise the NLSs, resulted in inhibition of the association of the pre-integration complex with karyopherin α and the subsequent inhibition of nuclear entry of the HIV-1 genome and infection.

ITI-367 and the oxadiazol class of compounds are selected using computer aided drug design (CADD) by modeling a 200,000 compound library on the crystal structure of the HIV-1 matrix protein. ITI-367 and the oxadiazol class of compounds were predicted to bind to the HIV-1 matrix protein within a structural groove in the N-terminal NLS generated by the inward orientation of tyrosine residue (amino acid #29). Previous compounds similarly selected by CADD for binding to the “tyrosine 29 pocket” were the subject of U.S. Pat. No. 5,849,793.

The class of compounds described herein have as a core structure the five sided ring structure (1,2,4-oxadiazol-5(4H)-one). In the examples presented below ITI-367 [3-(2-methoxyphenyl)-4-[3-(trifluoromethyl)phenyl-1,2,4-oxadiazol-5(4H)-one] has two aromatic groups (2-methoxyphenyl) and (3(trifluromethyl)-phenyl) associated with the ITI-367 structure.

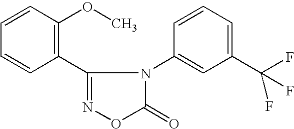

The present invention also provides a compound having a structure within the general formula: 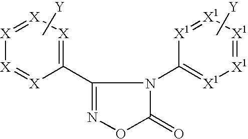
wherein X and X¹ are nitrogen or carbon insofar as at least three of X and at least three of X¹ are carbon; and when either X or X¹ are carbon, Y is covalently bonded to X or X¹, Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C ₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO₂, or —O—CH₃.

In other embodiments, the invention is directed to a compound wherein X and X¹ are nitrogen or carbon insofar as at least three of X and at least three of X¹ are carbon; and when X or X¹ are carbon, Y is covalently bonded to X or X¹ and is either 1) hydrogen or 2) chlorine, fluorine, —NO₂, —CF₃, —CH₃, or —O—CH₃.

In more specific embodiments, the invention is directed to a compound wherein X and X¹ are N or C; and when C, Y is covalently bonded to X or X¹ and is either 1)H or 2) —NO₂, Cl, F, —CF₃, —CH₃, or —O—CH₃.

The present invention is directed to each distinct variant disclosed in the general structure set forth above. For instance, in one embodiment, all of X and X¹ are C and Y is H in all. In other embodiments, only one of X and/or X¹ is N (as in a pyridine ring), or O or S; and the remaining of X and X¹ are C that may have Y or H as a substituent as Y is defined above.

Any combination of carbon or nitrogen in X and X¹ is encompassed by the invention. For instance two of X or X¹ may be nitrogen on each ring at positions ortho, meta or para to each other. In some embodiments, X will have two nitrogens, and X¹ will have zero, one or two nitrogens. In other embodiments, X will have one nitrogen and X¹ will have zero, one or two nitrogens. In any of these embodiments, where a ring has two nitrogens, on either ring these nitrogens may occur at positions ortho, para or meta to each other on the ring.

In other embodiments, the ring containing X¹ may have two nitrogens at positions ortho, para or meta on the ring, while the ring containing X may have zero, one or two nitrogens at positions ortho, para or meta on that ring.

In all the above combinations if any X or X¹ is a carbon, it may be unsubstituted (i.e., covalently bonded with hydrogen as the substituent) or substituted (i.e., covalently bonded with another substituent than hydrogen) as noted above.

In any “substituted” embodiment, as described in this specification, the substituent other than hydrogen can be any functional group defined within this disclosure, including the specific functionalities described in the markush groups above.

The methods of organic synthesis of ITI-367 are well-known in the art to the organic chemist. Methods of synthesizing compounds within the scope of the general formula can be determined by one of ordinary skill in the art without undue experimentation when guided by the teaching of the specification as to forming the specific chemical structure of any given compound within the general formula.

Specific additional examples of compounds according to the invention are listed in TABLE 1 that follows.

TABLE 1

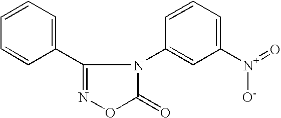

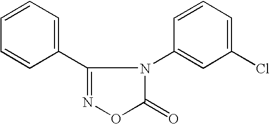

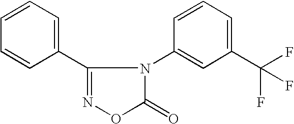

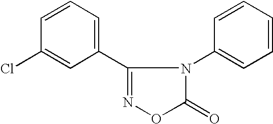

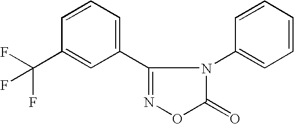

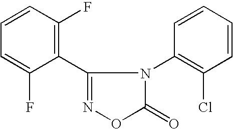

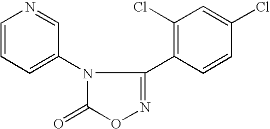

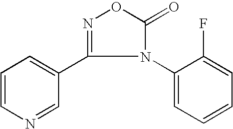

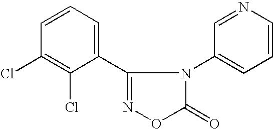

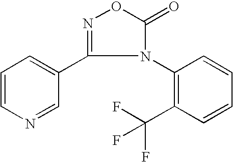

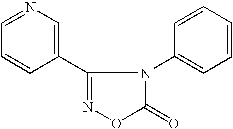

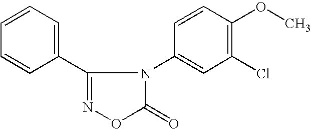

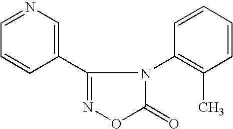

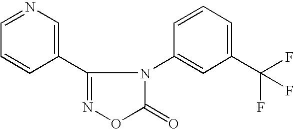

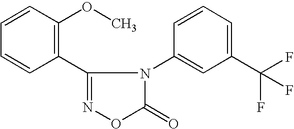

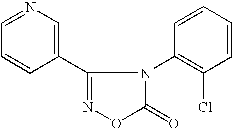

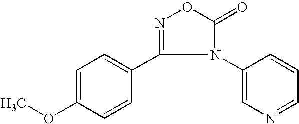

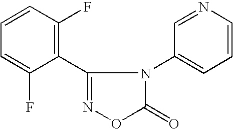

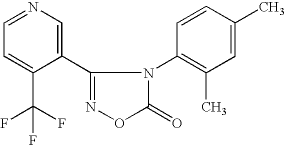

The present invention further provides a pharmaceutical composition comprising a compound of the above formula and a pharmaceutically acceptable carrier. The present invention also provides a method for treating HIV infection, comprising administering an effective amount of a compound according to the general structure above.

The compounds can be synthesized by common organic synthesis techniques. The inventive pharmaceutical complex or inventive pharmaceutical combination can be administered to a patient either by itself (complex or combination) or in pharmaceutical compositions where it is mixed with suitable carriers and excipients. The inventive compound or pharmaceutical composition can be administered parenterally, such as by intravenous injection or infusion, intraperitoneal injection, subcutaneous injection, or intramuscular injection. The inventive compound or pharmaceutical composition can be administered orally or rectally through appropriate formulation with carriers and excipients to form tablets, pills, capsules, liquids, gels, syrups, slurries, suspensions and the like. The inventive compound or pharmaceutical composition can be administered topically, such as by skin patch, to achieve consistent systemic levels of active agent. The inventive compound or pharmaceutical composition is formulated into topical creams, skin or mucosal patch, liquids or gels suitable to topical application to skin or mucosal membrane surfaces. The inventive compound or pharmaceutical composition can be administered by inhaler to the respiratory tract for local or systemic treatment of HIV infection.

The compounds and methods of the invention can be practiced in combination regimens with a Nucleoside analog Reverse Transcriptase inhibitors (NRTi), such as AZT, ZDV ddl, ddc, ddc, 3TC, abacavir; a Non-Nucleoside analog Reverse Transcriptase inhibitors (NNRTi) such as Nevirapine, Delavirdine, Efavirenz, Capravirine, Calanolide-A; a Protease inhibitors (Pi) such as SQV, RTV, IDV, NFV, APV, LPV, ATZ, FPV, or TPV or the new class of drugs known as Cell Entry inhibitors (Ci). These Ci inhibitors include Fuzeon, T-1249, PRO-542, and SCH-C. Only one cell entry inhibitor has been approved by the U.S. Food and Drug Administration (FDA): Fuzeon™ (T-20). This drug targets the gp41 protein on HIV's surface. T-1249, an entry inhibitor that is being developed by the same manufacturers of Fuzeon, also targets the gp41 protein. PRO-542 targets the CD4 protein on T-cells and SCH-C targets the CCR5 protein on T-cells.

The dosage of the inventive compound or pharmaceutical composition suitable for use with the present invention can be determined by those skilled in the art from this disclosure. The pharmaceutical composition will contain an effective dosage (depending upon the route of administration and pharmacokinetics of the active agent) of the inventive compound or pharmaceutical composition and suitable pharmaceutical carriers and excipients, which are suitable for the particular route of administration of the formulation (i.e., oral, parenteral, topical or by inhalation). The active compound is mixed into the pharmaceutical formulation by means of mixing, dissolving, granulating, dragee-making, emulsifying, encapsulating, entrapping or lyophilizing processes. The pharmaceutical formulations for parenteral administration include aqueous solutions of the active complex or combination in water-soluble form. Additionally, suspensions of the active compound may be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. The suspension may optionally contain stabilizers or agents to increase the solubility of the complex or combination to allow for more concentrated solutions.

Pharmaceutical formulations for oral administration can be obtained by combining the active compound with solid excipients, such as sugars (e.g., lactose, sucrose, mannitol or sorbitol), cellulose preparations (e.g., starch, methyl cellulose, hydroxypropylmethyl cellulose, and sodium carboxymethyl cellulose), gelatin, gums, or polyvinylpyrrolidone. In addition, a disintegrating agent may be added, and a stabilizer may be added.

Experimental Methods

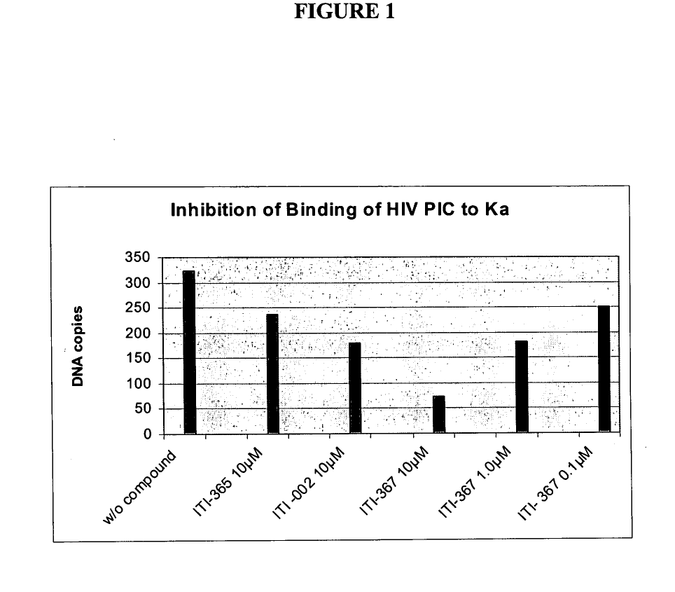

The inhibition of a molecular target by ITI-367 is illustrated in FIG. 1. The molecular target for nuclear entry of HIV-1 is the association of the viral matrix protein (p17) carrying the nuclear localization sequences, with the intracellular protein karyopherin α. To validate the activity of ITI-367, a cell free assay was used whereby human monocyte-derived macrophages collected from fresh blood samples were infected with HIV-1. At the predetermined time cells were lysed and the cytoplasmic extracts collected. The extracts contain HIV-1 pre-integration complexes (PIC) that represent the structure within which the HIV-1 cDNA is imported into the nucleus. The PIC is comprised of a number of viral proteins including MA (p17). Compounds that bind to MA and inhibit its association with Kα will also inhibit the association of the PIC with Kα. Therefore, this is a cell free assay. The extent of binding was measured by evaluating the levels of HIV DNA found in the Kα fraction in the presence or absence of test compound.

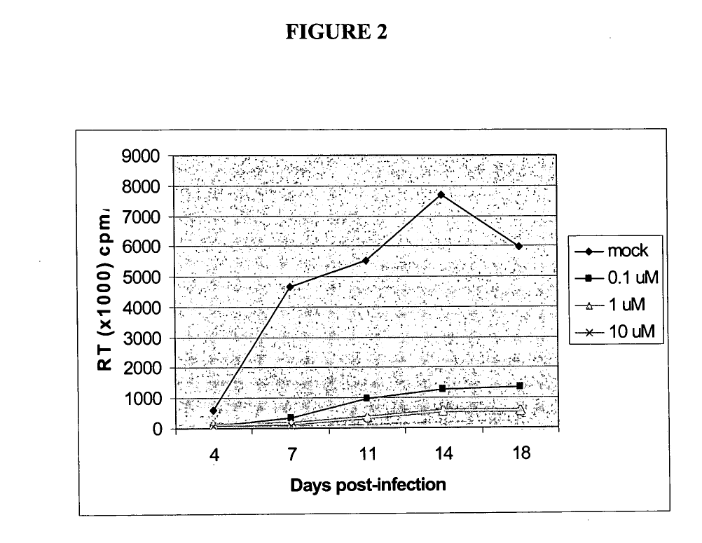

In FIG. 2 the inhibition of HIV-1 infection in monocyte-derived macrophages by ITI-367 is illustrated. Macrophages were prepared as described above. After a week of culture in M-CSF, cells were washed several times in RPMI and pre-incubated in complete (NHS, pen/strep, L-glutamine) medium with compound ITI-367 in appropriate concentration overnight (10, 1, 0.1, and 0.01 μM). [ITI-367 having been dissolved in DMSO at 10 mM concentration which was used as a stock solution (stored at −70° C.). From the stock solutions a 50 μM solution of compound in RPMI was prepared and stored at 4° C.]. After overnight incubation with compound, cells were infected with 500,000 cpm (RT activity) of the ADA strain of HIV-1 in 500 μl of RPMI with or without the compound. Two hours after infection, cells were washed with RPMI and incubated in fresh complete medium with the appropriate concentration of compound. Every 3-4 days, half of the medium was collected and used to quantify the level of virus production. Cultures were supplemented with additional medium. Infected macrophages were maintained for 2-3 weeks.

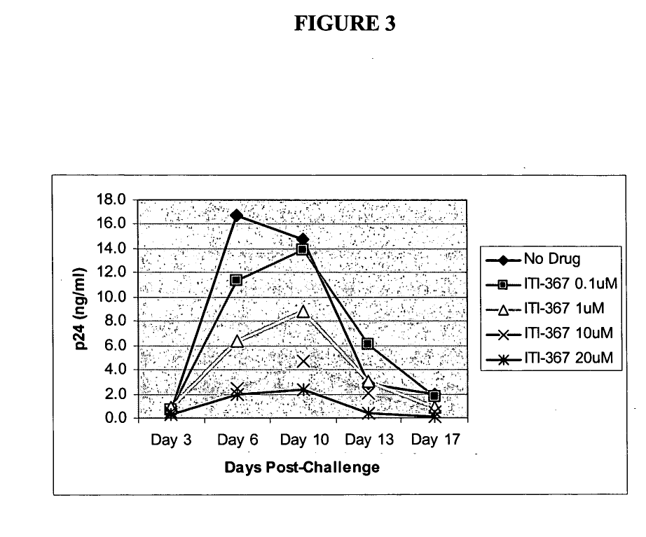

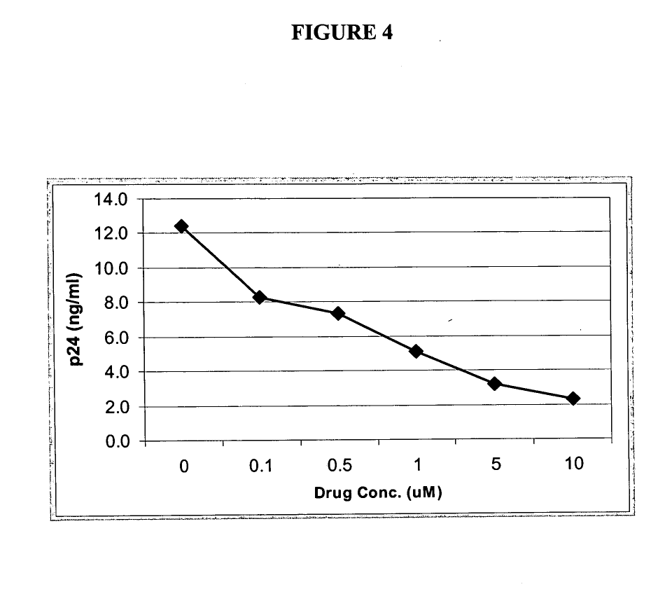

The inhibition of HIV-I infection of T lymphocytes by ITI-367 is shown in FIG. 3 and FIG. 4. T lymphocytes were isolated from tonsil tissue 24 hours post-surgical removal and then frozen. Aliquots of all were thawed on day of testing. There was no exogenous stimulation of the tonsil cells. The virus was a CCR5 clinical isolate at an approximate MOI of 1:40,000. Cells were incubated in the presence of ITI-367 for 19 hours prior to a 2.5 hour infection, at which time the virus inoculum was removed followed by two complete media exchanges. Cell culture media was Iscove's Media with 2 mM L-Glutamine, 100U/ml Penicillin, 100 ug/ml Streptomycin, and 8% heat-inactivated human serum AB. The drug concentrations used were 20 uM, 10 uM, 1 uM and 0.1 uM, prepared from a 5 mM DMSO stock. A no drug infection control (media only) was also evaluated in parallel. Replicates of four or six cultures were used to assess virus production from ITI-367 treated or control samples, respectively. Virus production was evaluated on days 3, 6, 10, 13, & 17 post-infection, by specific ELISA for HIV-1 core antigen (p24). Also, in FIG. 4, virus production was evaluated as a function of drug concentrations at 0, 0.1, 0.5, 1, 5 and 10 μM.

ITI-367 in Combination Assay

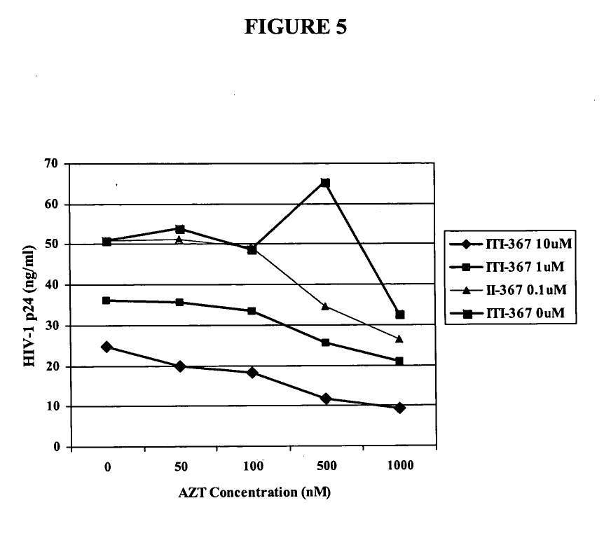

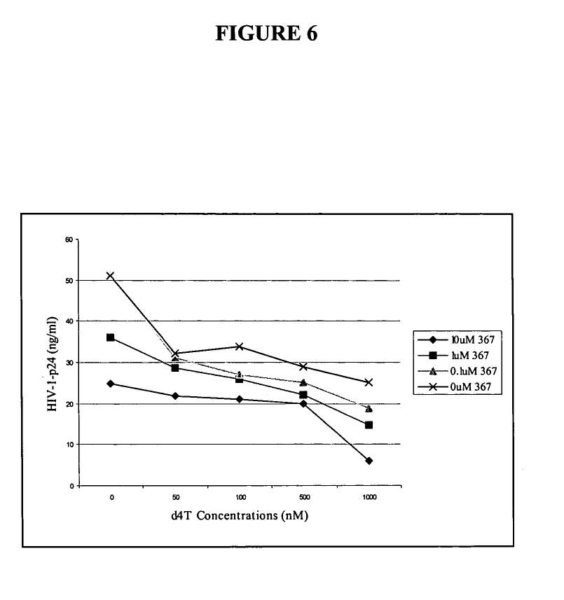

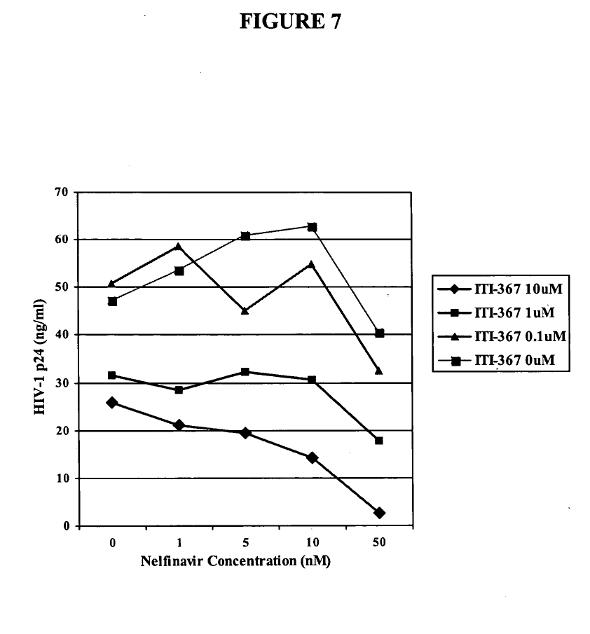

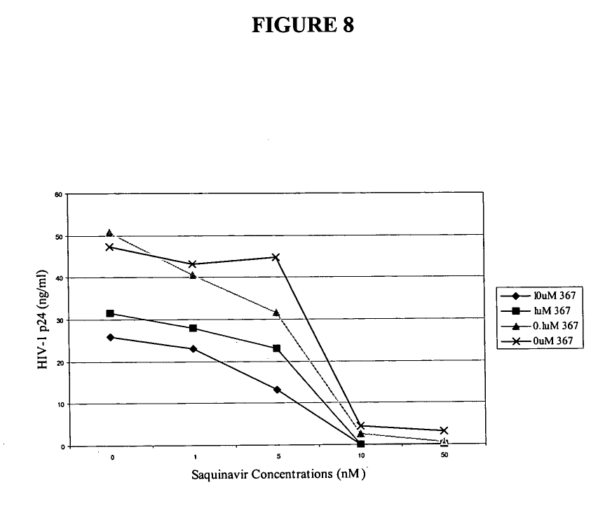

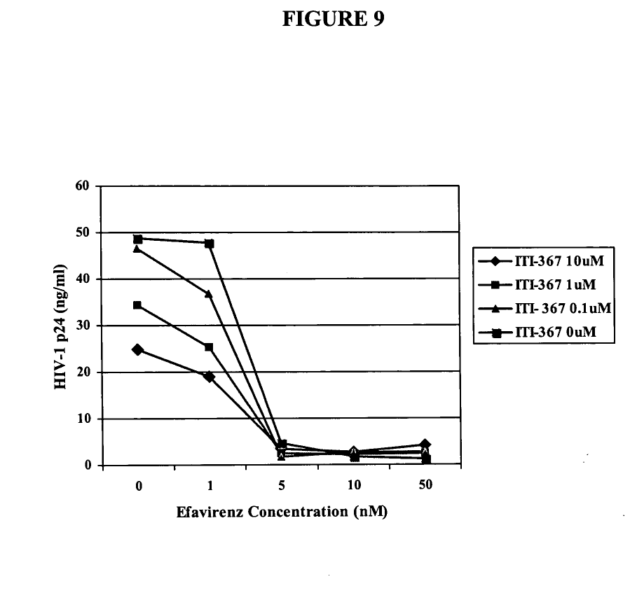

The inhibitory potential of ITI-367 was tested using HIV-1 infectivity assays in combination with different FDA-approved drugs. The data demonstrates the synergism of ITI-367 when used in combination therapy to treat HIV-1. Tonsil cells, isolated 24 hours post-surgical removal of tonsil tissue then frozen, were used as the target cells. Aliquots of the cells were thawed on the day of testing. There was no exogenous stimulation of the tonsil cells. The virus was a CCR5 clinical isolate at an approximate MOI of 1:40,000. Cells were incubated in the presence of drug for 17 hours prior to a 3.5 hour infection, at which time the virus inoculum was removed through three complete media exchanges. The cells were in the presence of drug before, during, and after all media removals. There were 2 FDA-approved drugs from each class of antiviral treatment; Nucleoside analogs Reverse Transcriptase Inhibitor (NRTi: AZT (FIG. 5), d4T(FIG. 6), Non-Nucleoside analogs Reverse Transcriptase Inhibitor (NNRTi: efavirenz (FIG. 9)), and Protease Inhibitor (Pi: saquinavir (FIG. 8), nelfinavir (FIG. 7)), were tested in combination with ITI-367. The RTi's were tittered over 4 concentrations (1000 nM, 500 nM, 100 nM, & 50 nM) and a No Drug Control (OnM), while the NNRTi's and Pi's were tittered over 50 nM, 10 nM, 5 nM, 1 nM, & OnM. All FDA drugs were prepared from lOmM DMSO stocks. ITI-367 was tested alone and in combination with each of the previously mentioned FDA-approved drugs. ITI-367 was tested at 10 μM, 1 μM, 0.1 μM, & 0 μM, prepared from a 100 μmM DMSO stock. The No Drug Controls were prepared using cell culture media only. Cells were split into 6×96-wells (No Drug Controls) and 2×96-wells (all 367+FDA-approved combinations) at ˜1.3E+06 cells/well and 200 ul volume. Cell culture media was Iscove's Media with 2 mM L-Glutamine, 100 U/ml Penicillin, 100 ug/ml Streptomycin, and 8% heat-inactivated human serum AB.

Cells were cultured at 37° C. and 5% CO₂. Samples (150 μl) were harvested on days 3, 5, 7, and 10 post-infection for HIV-1 p24 content analysis by ELISA. Cultures were supplemented with 150 μl of culture media containing the appropriate concentrations of ITI-367. Uninfected, untreated cells were counted at each sampling to monitor tonsil cell growth kinetics.

Results

ITI-367 exhibited a synergistic effect on inhibition of HIV-1 replication when combined with any of the three classes of approved HIV-1 therapeutics that currently comprise the Highly Active Anti-Retroviral Therapy (HAART). These results demonstrate that ITI-367, and related inhibitors as shown in the general chemical structure above will exhibit similar effects when combined with the class of HIV-1 therapeutics referred to as cell entry inhibitors as a synergistic effect is observed with all other classes.

From the foregoing description, it can be seen that the present invention comprises new and unique compounds, compositions, methods of making the compounds and compositions and methods of using the compounds and compositions resulting therefrom. It will be recognized by those skilled in the art that changes could be made to the above-described embodiments of the invention without departing from the broad inventive concepts thereof. It is understood, therefore, that this invention is not limited to the particular embodiments disclosed, but is intended to cover all modifications which are within the spirit and scope of the invention and that this invention is not limited to the particular embodiments disclosed, but it is intended to cover any modifications which are within the spirit and scope of the present invention as defined by the appended claims.

1. A compound having a structure within the general formula: 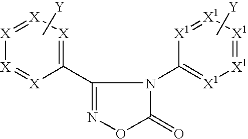
wherein X and X¹ are nitrogen or carbon insofar as at least three of X and at least three of X¹ are carbon; and when either X or X¹ are carbon, Y is covalently bonded to X or X^{1,} Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO₂, or —O—CH₃, and excluding the following compounds:

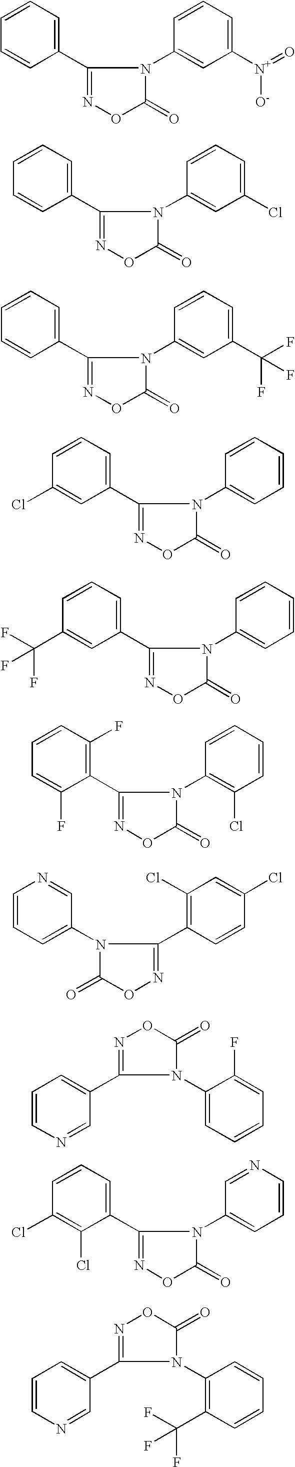

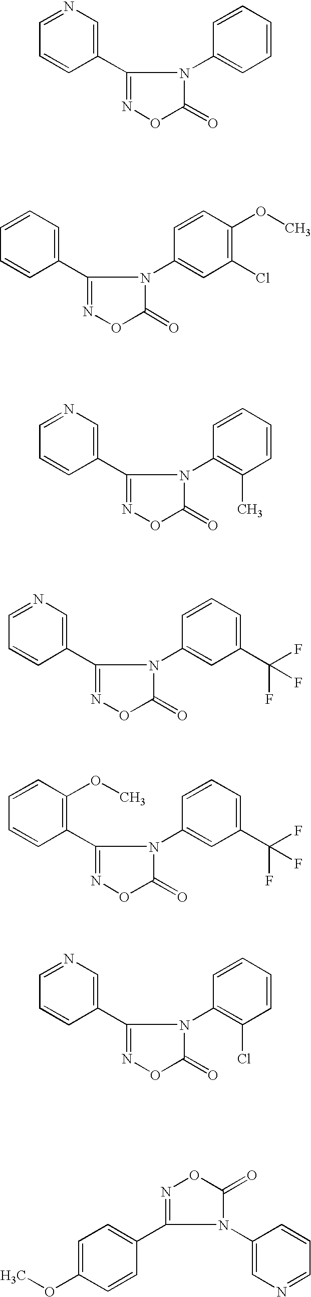

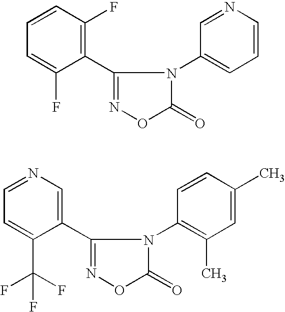

2. A compound according to claim 1, wherein X and X¹ are nitrogen or carbon insofar as at least three of X and at least three of X¹ are carbon; and when X or X¹ are carbon, Y is covalently bonded to X or X¹ and is either 1) hydrogen or 2) chlorine, fluorine, —NO₂, —CF₃, —CH₃, or —O—CH₃ .

3. A combination comprising a compound within the general formula: 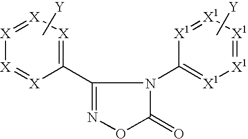
wherein X and X¹ are nitrogen or carbon insofar as at least three of X and at least three of X¹ are carbon; and when either X or X¹ are carbon, Y is covalently bonded to X or X¹, Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO₂, or —O—CH₃, and
further comprising at least one inhibitor compound selected from the group consisting of Nucleoside analog Reverse Transcriptase inhibitors (NRTi), Non-Nucleoside analog Reverse Transcriptase inhibitors (NNRTi), Protease inhibitors (Pi), and Cell Entry inhibitors (Ci).

4. A pharmaceutical composition, comprising a pharmaceutical carrier and a compound having the formula: 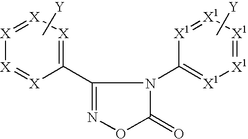
wherein X and X¹ are nitrogen or carbon insofar as at least three of X and at least three of X¹ are carbon; and when either X or X¹ are carbon, Y is covalently bonded to X or X^{1,} Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO₂, or —O—CH₃.

5. A composition according to claim 4, wherein X and X¹ are nitrogen or carbon insofar as at least three of X and at least three of X¹ are carbon; and when X or X¹ are carbon, Y is covalently bonded to X or X¹ and is either 1) hydrogen or 2) chlorine, fluorine, —NO₂, —CF₃, —CH₃, or —O—CH₃.

6. A composition according to claim 4, and further comprising at least one inhibitor compound selected from the group consisting of Nucleoside analog Reverse Transcriptase inhibitors (NRTi), Non-Nucleoside analog Reverse Transcriptase inhibitors (NNRTi), Protease inhibitors (Pi), and Cell Entry inhibitors (Ci).

7. A method of making a compound comprising synthesizing a compound within the general formula: 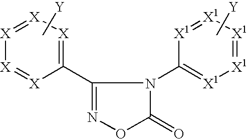
wherein X and X¹ are nitrogen or carbon insofar as at least three of X and at least three of X¹ are carbon; and when either X or X¹ are carbon, Y is covalently bonded to X or X^{1,} Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO₂, or —O—CH₃, with the proviso that when A is a halogen, n is 0-2.

8. A method of making a compound according to claim 7, wherein X and X¹ are nitrogen or carbon insofar as at least three of X and at least three of X¹ are carbon; and when X or X¹ are carbon, Y is covalently bonded to X or X¹ and is either 1) hydrogen or 2) chlorine, fluorine, —NO₂, —CF₃, —CH₃, or —O—CH₃.

9. A method of making a compound according to claim 7, wherein X and X¹ are nitrogen or carbon insofar as at least four of X and at least three of X¹ are carbon; and when X or X¹ are carbon, Y is covalently bonded to X or X¹ and is either 1) hydrogen or 2) chlorine, fluorine, —NO₂, —CF₃, —CH₃, or —O—CH₃.

10. A method for treating HIV infection, comprising administering an effective amount of one or more compound(s) having the formula: 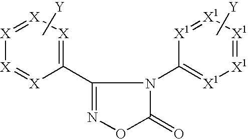
wherein X and X¹ are nitrogen or carbon insofar as at least three of X and at least three of X¹ are carbon; and when either X or X¹ are carbon, Y is covalently bonded to X or X^{1,} Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO₂, or —O—CH₃.

11. A method according to claim 10, wherein in said one or more compounds, X and X¹ are nitrogen or carbon insofar as at least three of X and at least three of X¹ are carbon; and when X or X¹ are carbon, Y is covalently bonded to X or X¹ and is either 1) hydrogen or 2) chlorine, fluorine, —NO₂, —CF₃, —CH₃, or —O—CH₃.

12. A method according to claim 10, one said compound having the formula:

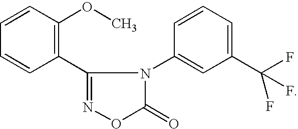

13. A combination therapeutic treatment regimen for the treatment of HIV infection, comprising a reverse transcriptase inhibitor and at least one compound having the formula: 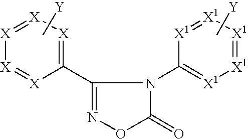
wherein X and X¹ are nitrogen or carbon insofar as at least three of X and at least three of X¹ are carbon; and when either X or X¹ are carbon, Y is covalently bonded to X or X^{1,} Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO_{2,} or —O—CH₃.

14. A combination according to claim 13, wherein X and X¹ are nitrogen or carbon insofar as at least three of X and at least three of X¹ are carbon; and when X or X¹ are carbon, Y is covalently bonded to X or X¹ and is either 1) hydrogen or 2) chlorine, fluorine, —NO₂, —CF₃, —CH₃, or —O—CH₃.

15. A combination according to claim 13, one said compound having the formula:

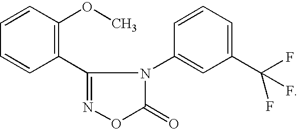

16. A method for treating HIV infection, comprising administering an effective amount of one or more compound(s) having a structure within the formula: 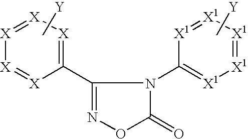
wherein X and X¹ are nitrogen or carbon insofar as at least three of X and at least three of X¹ are carbon; and when either X or X¹ are carbon, Y is covalently bonded to X or X^{1,} Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO₂, or —O—CH_{3,} and a reverse transcriptase inhibitor.

17. A method according to claim 16, wherein X and X¹ are nitrogen or carbon insofar as at least three of X and at least three of X¹ are carbon; and when X or X¹ are carbon, Y is covalently bonded to X or X¹ and is either 1) hydrogen or 2) chlorine, fluorine, —NO2, —CF₃, —CH₃, or —O—CH₃.

18. A method according to claim 16, one said compound having the formula:

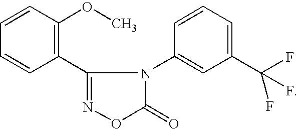

19. The combination of claim 13, wherein the reverse transcriptase inhibitor is selected from the group consisting of 3TC, AZT, ddI, d4T, ddC, and combinations thereof.

20. The combination of claim 13, further comprising an HIV protease inhibitor.

21. The combination of claim 20, wherein the HIV protease inhibitor is selected from the group consisting of ritonavir, nelfinavir, saquinavir, indinavir, and combinations thereof.

22. The method of claim 16, wherein the reverse transcriptase inhibitor is selected from the group consisting of 3TC, AZT, ddI, d4T, ddC, and combinations thereof.

23. The method of claim 16, further comprising an HIV protease inhibitor.

24. The method of claim 23, wherein the HIV protease inhibitor is selected from the group consisting of ritonavir, nelfinavir, saquinavir, indinavir, and combinations thereof.

25. A method of making a pharmaceutical composition comprising combining a pharmaceutical carrier with a compound having the formula: 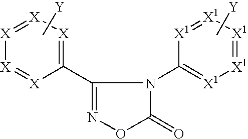
wherein X and X¹ are nitrogen or carbon insofar as at least three of X and at least three of X¹ are carbon; and when either X or X¹ are carbon, Y is covalently bonded to X or X^{1,} Y being either 1) hydrogen or 2) halogen, cyano, hydroxyl, thiol, sulfamoyl, alkoxyl, nitro, haloalkyl, alkyl, substituted alkyl, aryl, substituted aryl, acyl, carboxyl, chlorine, bromine, iodine, fluorine, nitroxyl, —R, —R(A)ₙ, —O—R(A)ₙ, or —S—R(A)ₙ; R being a straight or branched C₁₋₁₂ alkyl or alkoxy, either saturated or unsaturated, wherein R may be substituted with A at any carbon in R, n being 0-3 for each carbon, and A, if not hydrogen, is chlorine, bromine, iodine, fluorine, —NO₂, or —O—CH₃.

26. A method of making a composition according to claim 25, wherein X and X¹ are nitrogen or carbon insofar as at least three of X and at least three of X¹ are carbon; and when X or X¹ are carbon, Y is covalently bonded to X or X¹ and is either 1) hydrogen or 2) chlorine, fluorine, —NO₂, —CF₃, —CH₃, or —O—CH₃.

27. A method of making a composition according to claim 25, said compound having the formula:

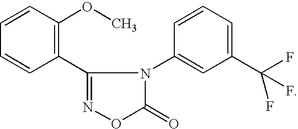

28. The combination according to claim 3, wherein the inhibitor compound is a Nucleoside analog Reverse Transcriptase inhibitor (NRTi).

29. The combination according to claim 3, wherein the NRTi is selected from the group consisting of AZT, ZDV, d4T, ddI, ddc, ddc, 3TC, and abacavir.

30. The combination according to claim 3, wherein the inhibitor compound is a Non-Nucleoside analog Reverse Transcriptase inhibitor (NNRTi).

31. The combination according to claim 3, wherein the NNRTi is selected from the group consisting of Nevirapine, Delavirdine, Efavirenz, Capravirine, and Calanolide-A.

32. The combination according to claim 3, wherein the inhibitor compound is a Protease inhibitor (Pi).

33. The combination according to claim 3, wherein the Pi is selected from the group consisting of SQV, RTV, IDV, NFV, APV, LPV, ATZ, FPV, and TPV.

34. The combination according to claim 3, wherein the inhibitor compound is a Cell Entry inhibitor (Ci).

35. The combination according to claim 3, wherein the Ci is selected from the group consisting of Fuzeon, T-1249, PRO-542, and SCH-C.

36. A compound having a structure within the general formula of claim 1, with the proviso that when A is a halogen, n is 0-2.

37. A compound having a structure within the general formula of claim 1, with the proviso that when at least one Y is halogen, the remaining of Y are not only either hydrogen or halogen.

38. A compound having a structure within the general formula of claim 1, with the proviso that when at least one Y contains a halogen, the remaining of Y are not only hydrogen.

39. A compound having a structure within the general formula of claim 1, with the proviso that all of Y are not only either hydrogen or alkyl.

40. A compound having a structure within the general formula of claim 1, with the proviso that when at least one Y is nitroxyl, the remaining of Y are not only hydrogen.

41. A compound having a structure within the general formula of claim 1, with the proviso that when at least one Y is an —O—R—(A)ₙ group, the remaining of Y are not only hydrogen.
